(19) United States
(12) Patent Application Publication
(10) Pub. No.: US 2004/0258673 A1
(43) Pub. Date: Dec. 23, 2004

(54) Elective collection and banking of autologous peripheral blood stem cells

(76) Inventors: Thomas Gordon Hirose, Huntington Beach, CA (US); Simon Sun-man Lam, Agoura Hills, CA (US); Rubio R. Punzalan, Torrance, CA (US); Denis O. Rodgerson, Malibu, CA (US)

Correspondence Address: LIU & LIU, 811 WEST SEVENTH STREET, SUITE 1100, LOS ANGELES, CA, 90017, US

(21) Appl. No.: 10/819,342

(22) Filed: Apr. 5, 2004

Related U.S. Application Data
(60) Provisional application No. 60/460,362, filed on Apr. 3, 2003.

(51) Int. Cl. A61K 48/00; C12N 5/08

(52) U.S. Cl. 424/93.21; 435/366

(57) ABSTRACT

An elective healthcare insurance model using an individual's own peripheral blood stem cells for the individual's future healthcare uses. An individual can elect to have his or her own stem cells collected, processed and preserved, while he or she is in healthy or “pre-disease” state, for future distribution for his or her healthcare needs. The process includes methods of collection, processing, preservation and distribution of adult (including pediatric) peripheral blood stem cells during non-diseased state. The stem cells collected will contain adequate dosage amounts, for one or more transplantations immediately when needed by the individual for future healthcare treatments. The collected adult or non-neonate child peripheral blood stem cells can be aliquoted into defined dosage fractions before cryopreservation so that cells can be withdrawn from storage without the necessity of thawing all of the collected cells.

This application claims the priority of provisional application No. 60/460,362, filed Apr. 3, 2003, which is fully incorporated by reference as if fully set forth herein.

### FIELD OF THE INVENTION

The present invention relates to stem cell collection and storage, and more particularly to the collection and storage of autologous peripheral blood stem cells.

### BACKGROUND OF THE INVENTION

Stem cell transplantations have been used either by itself (e.g. for congenital diseases) or in conjunction with other treatments such as chemotherapy for treating various diseases such as cancer. However, most of these stem cell transplants either use stem cells from matched donors (allogeneic) or stems cells collected from the patients (autologous) immediately before their treatment). In allogeneic transplantations, there are number of drawbacks such as immune rejections and graft-versus-host-diseases. In addition, allogeneic transplantation is much more expensive than autologous transplantation. Stem cells collected from umbilical cords may be used for some treatments, but the low cell dose, immaturity and incomplete complement of cells limit the immediate use of these stem cells for some treatments. Stem cells collected from the individual right before their treatment may contain contaminated cells from the disease being treated. This invention addresses various problems associated with the prior arts by providing a method and facility to collect, process, and store, and distribute healthy stem cells for future treatments of an individual's healthcare needs arise.

### SUMMARY OF THE INVENTION

This invention provides an elective healthcare insurance model using an individual's own peripheral blood stem cells for the individual's future healthcare uses. More specifically, this invention provides a method in which an individual can elect to have his or her own stem cells collected, processed and preserved, while he or she is in healthy state (at a time with no immediate perceived health condition requiring treatment using his own stem cells), for future distribution for his or her healthcare needs. The invention also embodies methods of collection, processing, preservation and distribution of adult (including pediatric) peripheral blood stem cells during non-diseased state. The stem cells collected will contain adequate dosage amounts, for one or more transplantations immediately when needed by the individual for future healthcare treatments.

In one aspect of the present invention, the current invention provides a cell bank to support an elective healthcare insurance model to effectively protect members of the population from future diseases. An individual can elect to have his or her own stem cells collected, processed and preserved, while he or she is in healthy state, for future distribution for his or her healthcare needs.

In another aspect of the present invention, this invention provides a method for collecting an adequate stem cell dosage from an individual donor during non-diseased state, processing the stem cells collected, cryogenically preserving them for future distribution for the donor's healthcare needs. As used in the context of the present invention described herein, the term “donor” refers to a person from whom stem cells are collected, intended for future treatment of that same person.

In one embodiment of the current invention, stem cells and progenitor cells are collected during the non-disease phase by the process of apheresis from adult or pediatric peripheral blood, processed to optimize the quantity and quality of the collected stem cells, cryogenically preserved, and used for autologous therapeutic purposes when needed after they have been thawed. Autologous therapeutic purposes are those in which the cells collected from the donor are infused into that donor at a later time. In particular, the present invention relates to the use of autologous adult or non-neonate child peripheral blood cells for the reconstitution of the donor's hemapoetic system, immune system or for repopulating areas in which cellular damage has occurred, with stem cells that will differentiate into cells of the same type as those damaged. Such cellular damage would include that caused by infarction of arteries of the heart, brain or other organs; or that caused by trauma, infection, or other factors. Hematopoietic reconstitution is an established therapy in a variety of diseases and disorders such as anemias; malignancies; immune and autoimmune deficiencies, disorders and dysfunctions; and neurological disease such as Parkinson's disease, Altzheimer's disease, and other neurological disorders. Hematopoietic reconstitution with autologous peripheral blood stem cells can occur with or without ablation of the bone marrow. Ablation is the destruction of bone marrow function by chemotherapy, ionizing radiation or a combination of chemotherapy and ionizing radiation. Such ablation can also occur as result of exposure to the ionizing radiation that is produced following a nuclear explosion. Autologous peripheral blood stem cells are considered curative for a high percentage of individuals exposed to lethal or sub-lethal levels of ionizing radiation.

In another embodiment, autologous adult or non-neonate child peripheral blood stem cells at doses below those used in the therapy of the above diseases and disorders, can be used without ablation to serve as boosters for the immune system in individuals to whom the immune system, is depressed due to illness, infections, stress, aging or other factors.

In a further embodiment, the present invention includes the processes such that the collected adult or non-neonate child peripheral blood stem cells can be aliquoted into defined dosage fractions before cryopreservation so that cells can be withdrawn from storage without the necessity of thawing all of the collected cells.

### BRIEF DESCRIPTION OF THE DRAWINGS

FIG. 1 is a flow diagram schematically representing the inventive process in accordance with one embodiment of the present invention.

FIG. 2 i s a flow diagram schematically representing the stem cell collection process in accordance with one embodiment of the present invention.

FIG. 3 is a flow diagram schematically representing stem cell processing in accordance with one embodiment of the present invention.

### DETAILED DESCRIPTION OF THE INVENTION

The present description is of the best presently contemplated mode of carrying out the invention. This description is made for the purpose of illustrating the general principles of the invention and should not be taken in a limiting sense. The scope of the invention is best determined by reference to the appended claims. This invention has been described herein in reference to various embodiments and drawings. It will be appreciated by those skilled in the art that variations and improvements may be accomplished in view of these teachings without deviating from the scope and spirit of the invention.

The present invention applies to all animals, in particular vertebrates. Examples of such vertebrates are mammals. An example of such a mammal is a human such as a human infant, child, or adult. For ease of discussion in this patent application, we use a human being (identified as a person or a donor) as a non-limiting example of such an animal. Also, for ease of discussion, the pronoun “he” is used. It is to be understood that the term “he” includes “he” and/or “she”. Following longstanding law convention, the terms “a” and “an” as used herein, including the claims, are understood to mean “one” or “more”.

### 1. Inventive Process

a. Overview

The present invention provides an elective healthcare insurance model using an individual's own peripheral blood stem cells for the individual's future healthcare uses. More specifically, this invention provides a method in which an individual can elect to have his or her own stem cells collected, processed and preserved, while he or she is in healthy state, for future distribution for his or her healthcare needs. The invention also embodies methods of collection, processing, preservation and distribution of adult (including pediatric) peripheral blood stem cells during non-diseased state. The stem cells collected will contain adequate dosage amounts, for one or more transplantations immediately when needed by the individual for future healthcare treatments.

b. Application of Present Invention

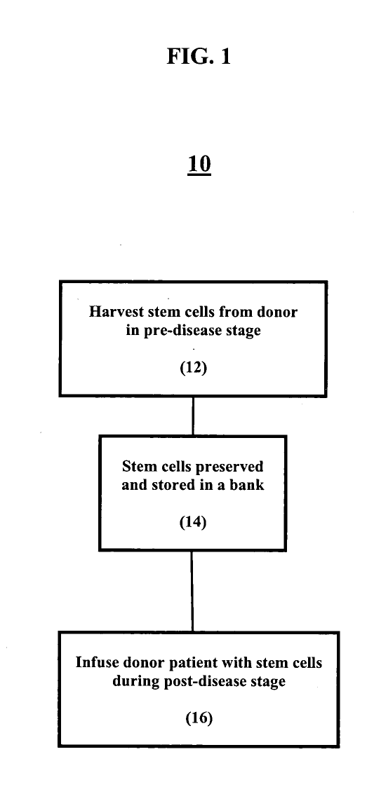

Referring to FIG. 1, the inventive process 10 in accordance with one embodiment of the present invention broadly comprises the following steps:

(1) Healthy stem cells are harvested from a donor in the “pre-disease” stage (12). The term “pre-disease” means indicate the state in which the donor is healthy, or before the donor has developed, manifested, or been diagnosed with any or a particular disease, which is known to affect the quality of the stem cells, or before the donor is deemed to be in a health condition that would render the donor not to be in a state qualified for stem cell collection.

(2) The pre-diseased cells are preserved and stored in a bank (14), e.g., by cryopreservation, for many years, such as for 500 years or less, or less than any number of years less than 500 years; or for at least a number of years before usage, such as at least 0 to 100 years, or at least any number of years therebetween.

(3) In the event that the donor develops, manifests, or is diagnosed with a disease (“post-disease” state), he is infused with the above previously preserved pre-disease cells (16). The term “post-disease” denotes the state at or after which the donor develops, has developed, manifests, has manifested, has been diagnosed with a disease, or his disease has become detectable or been detected.

It should be noted that the above discussion over “pre-disease” state (versus “post-disease” state) covers the absolute term of “healthy/no disease” (versus “not healthy/diseased”) and a relative term of a gradation in the disease progression (“healthier than” or “less diseased” than post-disease state). Since “pre-disease” can be defined by a time prior to a person being diagnosed with a disease, he could be healthy in an absolute term or he might already have the disease but only that it has not manifested itself, been diagnosed or detected. Even in the latter scenario, for such a “pre-disease” state, it is possible that the disease may not be so widespread such that it has reached the cells collected; or even if the cells collected are diseased, they may be less aggressive or are of a healthier grade due to the early stage of their development, or the cells still retain some functioning necessary to combat the same disease and/or other diseases. Thus, the term “healthy” cells covers both the absolute term that the cells are healthy, and the term that, relatively speaking, these collected cells (from the donor before he becomes a patient) are healthier than what the patient (in his “post-disease” state) currently have in his body.

Specifically, “pre-disease” state could refer to prior diagnosis or knowledge of a specific targeted disease or diseases, or class or classes of diseases, of the donor (collectively “specific diseases”), such that stem cell can be collected from the donor at an opportune time in anticipation of the donor manifesting the specific diseases in the future. For example, in view of family healthy history, genetic history and/or profiling, a donor may be deemed to have a certain probability of contracting a certain specific disease (e.g., a certain cancer) during adult years. In accordance with the process provided by the present invention, stem cells are collected from the donor during his early years before the disease manifests itself, which stem cells are banked in anticipation of this specific disease in the future. The donor may be subject to a medical examination to confirm that he is “pre-disease” with respect to the specific disease. At the time of stem cell collection, the donor may be diagnosed to have a disease or diseases, or class or classes of diseases different from the specific disease, which diagnosed diseases may be acceptable with respect to stem cell collection and/or the specific disease as perceived by prevailing medical practices. In other words, stem cells may be collected at a stage when the donor may actually posses, or be diagnosed with, a health condition that is not similar to the disease to be treated by stem cell transplantation.

Other definitions of “pre-disease” state may be adopted without departing from the scope and spirit of the present invention. For example, certain standards may be established to pre-diagnose the stem cell donor as being in a “pre-disease” state. This type of pre-diagnosis may be established as an optional screening process prior to collection of stem cells from the donor in the “pre-disease” state. Such “pre-disease” state standards may include one or more of the following considerations or references prior to collection, such as (a) pre-specific disease; (b) prior to actual knowledge by donor and/or health professionals of specific or general diseases; (c) prior to contraction and/or diagnosis of one or more classes of diseases; (d) prior to one or more threshold parameters of the donor relating to certain diseases, for example at a certain age, with respect to certain physical conditions and/or symptoms, with respect to certain specific diseases, with respect to certain prior treatment history and/or preventive treatment, etc.; (e) whether the donor fits into one or more established statistical and/or demographic models or profiles (e.g., statistically unlikely to acquire certain diseases); and (f) whether the donor is in a certain acceptable health condition as perceived based on prevailing medical practices.

c. Advantage of the Present Invention

The present invention presents methods for using autologous stem cell transplants, such as those from peripheral blood, and bone marrow from post-birth human (including baby, child and adult), for the treatment of diseases. In a non-limiting example of the invention, the diseases treated are cancer and immunodiseases such as acquired immunodeficiency syndrome (AIDS). The invention has an advantage over umbilical cord blood transplants since for the overwhelming majority of children and adult, their umbilical cord blood at birth is no longer available.

The advantages of the present invention includes the following:

(1) Since the method uses autologous transplant, there is no need to expend time, money and energy in “cleansing” the SC or donor marrow of potentially dangerous mature T cells (thought to be important for the development of graft-versus-host-disease). This is especially advantageous because such cleansing process may introduce harmful impurities into the bone marrow. Examples of cleansing process are: chemicals or monoclonal antibody (OKT3) that specifically recognizes and eliminate mature T cells.

In the case of patients with certain existing diseases other than the targeted disease at the time of collection, the “healthy” harvested cells will not contain the disease vectors of the targeted disease, such as in the case of AIDS patients, the pre-disease cells will not contain the AIDS virus.

(2) Since the method uses autologous transplant, there is no need to laboriously locate SC or bone marrow from another donor or to conduct tests to ensure that the SC or bone marrow matches that of the recipient. Further, there is no fear that matching SC or bone marrow may not be found or that crucial time is lost to test and locate such matching SC or bone marrow such that the recipient may be in mortal danger or incur fatal injury by the time a match is found.

(3) Since the method uses autologous transplant, there is no fear of graft-versus-host disease; or immune rejection.

(4) Autologous stem cells do not carry the risk of infectious disease found when one person's stem cells are used for another person.

(5) Since the method uses autologous transplant, there is no fear of transplant transmitted disease (e.g. HIV, CMV, hepatitis, syphilis etc.)

(6) Of particular importance is the fact that these autologous transplants are harvested prior to the development of disease (pre-disease stage) as compared to the period of time after disease occurs (post-disease stage). The pre-disease state autologous stem cell transplant has the following advantages over transplantation using autologous stem cells harvested after disease occurs:

(i) The previously harvested pre-disease cells will be younger. Among the possible advantages associated with youth are: the cells will likely to be more resilient, more versatile, and would retain normal (or relative more normal) activities and a full range of (or broader range of) activities, and thus more well-equipped and more vigorous in combating a disease, as compared to stem cells collected after disease occurs. Further, due to advance in age (e.g., in old age), certain population of cells may be depleted, missing or no longer be available for harvesting at a later stage in a human's life. Also, certain cellular functions or genes may be turned off in older cells, down-regulated, or lost, due to the natural aging process, aged related deterioration, mutation, or accumulated “wear-and-tear”, or environmental assaults over the years, etc. Further, older cells (post-disease versus pre-disease cells) may contain more mutations, defects, due to age or mistakes in the replication process, or environment assaults.

(ii) The previously harvested (pre-disease) cells from the donor, prior to him becoming a patient, will be healthy cells (or healthier than the current cells existing in the patient) and may be more easily grown or programmed, or more readily programmable than the post-disease cells.

(iii) The pre-disease population of the harvested cells will be healthy or will contain more healthier (or less diseased) cells than the post-disease population of cells, and thus the population of the pre-disease harvested cells will not be contaminated or be less contaminated by diseased cells which may be re-introduced into the patient and potentially cause a relapse.

For example, in the case of cancer treatment, the present invention has the following advantages over the prior art described above. The infused cells will not be contaminated with cancer cells (or will be less contaminated with cancer cells if the collection occurred after the disease has taken hold but before its diagnosis) as compared to the cells collected from a patient who has already developed cancer. Therefore, no laborious, time-consuming, inefficient methods (that may even inadvertently introduce undesirable chemicals) assays and screenings are required to cleanse the harvested cell population to remove cancer cells from the pre-disease harvested cells. Further, the present invention eliminates or reduces the possibility of causing a relapse through infusion of sub-optimal cancer cell depicted stem cell products.

(iv) The population of harvested cells may be more “well-rounded” or more normal/healthy in that a full range of normally occurring cells will be present relative to older diseased cell population. For example, the peripheral blood SC collected from an AIDS patient will be deficient in T helper cells which are decimated by the AIDS virus; but found in normal number in the previously collected and healthy population of cells.

(v) Furthermore, hematopoietic stem and progenitor cells can potentially be multiplied in culture, before or after cryopreservation, thus expanding the number of stem cells available for therapy. Thus, the population of healthy cells may be increased by cellular expansion, and infused into the patient to greatly boost his immunodefense in the number of cells available and that the cells are healthy.

(vi) Furthermore, processed hematopoietic stem cells may undergo immuno modulation or cellular adaptation inherent in the processing and cryopreservation technique which may improve the stem cell product.

Various stages of the inventive process are discussed in greater detail below.

### 2. Stem Cell Collection Process

The inventive aspect of the step cell collection process is directed to the timing and the health state of the individual when the collection occurs. Under this invention, the collection process occurs when the individual is in non-diseased state. The physical steps of collecting stem cells may comprise those steps known in the art.

Stem cells are primarily found in the insides of long bones (legs, hips, sternum etc.) and comprise the “bone marrow”. These stem cells may leave the bone marrow and circulate in the blood stream. Stem cells comprise approximately 0.1-1.0% of the total nucleated cells as measured by the surrogate CD34+ cells, and may be collected using a machine called an apheresis instrument. Many hundreds of thousands of apheresis collections take place each year for platelets, red cells, plasma and stem cells. It has been shown to be safe and effective technology.

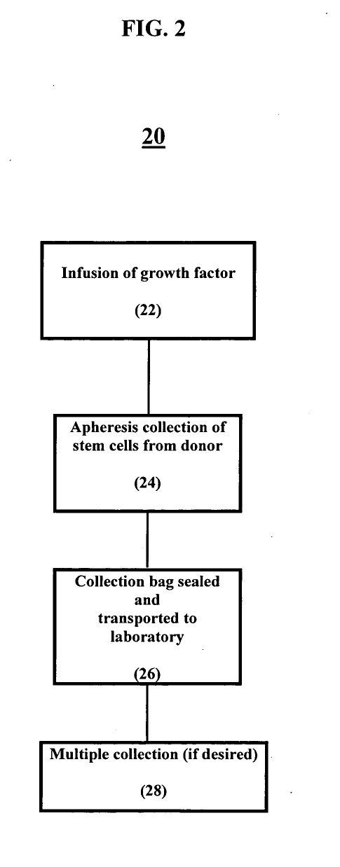

FIG. 2 schematically illustrates the steps involved in the stem cell collection process (20) in accordance with one embodiment of the present invention. The amount of stem cells circulating in the peripheral blood cell may be increased with the infusion of cell growth factors (22), called granulocyte colony stimulating factor (GCSF) prior to collection. The infusion of growth factors is routinely given to bone marrow and peripheral blood donors and has not been associated with any long lasting untoward effects. Adverse side effects are not common but include the possibility of pain in the shin, mild headache, mild nausea and a transient elevation in temperature. The growth factor is given 1-6 days before peripheral blood stem cells are collected.

1-6 days after GSCF is infused the peripheral blood stem cells are sterilely collected by an apheresis instrument (24). The apheresis instrument looks very much like a dialysis machine, but differs in that it is a centrifuge while a dialysis machine uses filtration technology. Stem cell collection can be accomplished in the privacy of the donors own home or in a collection center. Blood is drawn from one arm then enters the apheresis instrument where the stem cells are separated and collected. The rest of the whole blood is then returned to the donor. A registered nurse (RN) places a needle into both arms of the donor in the same manner as a routine blood collection. The RN then operates the apheresis instrument that separates the blood elements (red cells, white cells, plasma) collecting the stem cells and returning the rest of the whole blood to the donor. The collection of stem cells requires approximately 2-4 hours during which the donor is relaxing and watching a movie. Shortly after the apheresis collection, the bone marrow releases more stem cells into the blood stream to replace the harvested stem cells. The amount of stem cells collected is a very small fraction of a person's stem cells and as thus the procedure does not deplete the body of stem cells.

Adverse medical reactions during apheresis collections are uncommon and may consist of a tingling sensation in the mouth and fingers. This reaction is usually mild in nature and does not stop the stem cell collection.

Following collection of the stem cells (26) the collection bag is sealed and then transported to the laboratory for processing, testing and cryopreservation. Stem cells may be transported by methods known in the art. For example, conventional containers for blood can be used for transport, e.g. thermally validated containers can be transported by express methods or messengers. In these embodiments, the temperature of the container remains essentially constant over long periods of time.

Depending on the situation and the quantity and quality of stem cells to be collected from the donor, it may be preferable to collect the stem cells from donors when they are at an “adult” or a “matured” age (the term “adult” as used herein refers to and includes adult and non-neonate, unless otherwise used in a particular context to take a different meaning) and/or at a certain minimum weight. For example, stem cell is collected when the donor is within a range from 10 to 200 Kg. in accordance with one embodiment of the present invention, or any range within such range, such as 20 to 40 Kg. In addition or in the alternative, it may be required that the donor be of a certain age, within a range from 2-80 years old in accordance with one embodiment of the present invention, or any range within such range, such as 9 to 18 years old, or 12 to 16 years old, or any range of ages within such age ranges, or as determined statistically. Certain legal requirements may also prescribe and/or limit the appropriate age and/or or weight of the donor for stem cell collection.

In one embodiment of the present invention, a donor may elect to have stem cell collection in multiple stages (28), to increase the amount of stem cells to be bank for future use. For example, he may elect to have stem cells collected at different age and/or weight. Different units of stem cells can be collected at each collection, as appropriate depending on the age and/or weight of the donor at the time of collection. Generally, more stem cells can be collected during a single collection process, as the age and/or weight of the donor increase. Further, in addition or in the alternative, he may elect to have stem cell collected pre-disease and post-disease (i.e., after the period of pre-disease as defined herein). Still further, in addition or in the alternative, he may elect to have stem cell collected periodically or at specified times pre-disease, independent of his weight and/or age, and to map the progress of the health condition of the donor.

### 3. Stem Cell Processing

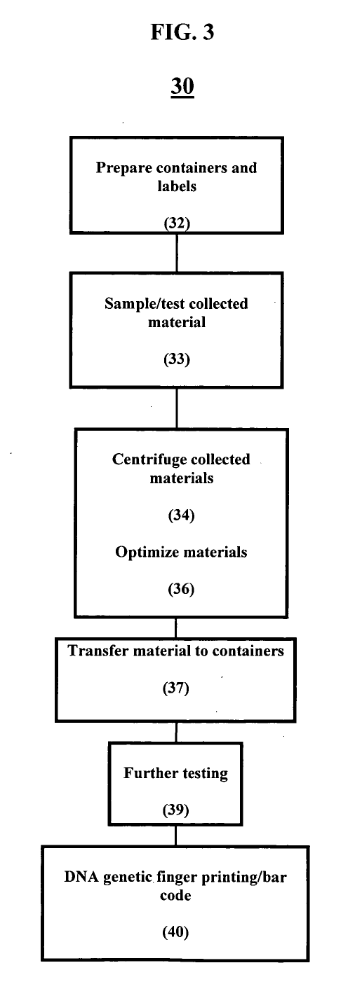

In some embodiments of the invention, after collection, the stem cells are processed according to methods known in the art (see, for example, Lasky, L. C. and Warkentin, P. I.; Marrow and Stem Cell Processing for Transplantation; American Association of Blood Banks (2002). In an embodiment of the invention schematically illustrated in FIG. 3, processing (30) may include the following steps: preparation of containers (e.g., tubes) and labels (32), sampling and/or testing of the collected material (33), centrifugation (34), transfer of material from collection containers to storage containers (37), the addition of cryoprotectant (38), etc. In some embodiments, after processing, some of the processed stem cells can be made available for further testing (39).

Specific uniqueness of this invention is that there will be no requirement for any kind of tissue typing since the collected stem cells will be used for autologous transplantation. However, tissue typing of specific kinds may be used for sample identification or for the use of these stem cells for possible allogeneic use. This type of information may include genotypic or phenotypic information. Phenotypic information may include any observable or measurable characteristic, either at a macroscopic or system level or microscopic, cellular or molecular level. Genotypic information may refer to a specific genetic composition of a specific individual organism, including one or more variations or mutations in the genetic composition of the individual's genome and the possible relationship of that genetic composition to disease. An example of this genotypic information is the genetic “fingerprint” and the Human Leukocyte Antigen (HLA) type of the donor. In some embodiments of the invention the stem cells will be processed in such a way that defined dosages for transplantation will be identified and aliquoted into appropriate containers.

In preferred embodiments, the number of cells collected in a single collection session may be equal or greater than 2×10⁹ total nucleated cells, or at least on the order of 10⁹, or 10⁸, or 10⁷, or 10⁶, or 10⁵ total nucleated cells, depending on the weight and age of the donor. Aliquoting of these cells may be performed so that a quantity of cells sufficient for one transplant (1×10⁹ total nucleated cells) will be stored in one cryocyte bag or tube, while quantities of cells appropriate for micro-transplantation (supplemental stem cell infusion), will be stored in 10 aliquots of 2×10⁸ total nucleated cells, in appropriate containers (cryocyte bags or cryotubes). Generally, at least one unit is collected at each collection session, and each unit collected is targeted at less than on the order of 10⁶ total nucleated cells per Kg. weight of the person, in accordance with one embodiment of the present invention. This process constitutes a unique process for “unitized storage” enabling individuals to withdraw quantities of cells for autologous use without the necessity of thawing the total volume of cells in storage (further details discussed below). This may include processing the harvested stem cells (36) to optimize the quantity of total nucleated cells to ensure sufficient number of cells for targeted diseases without or with little waste of cells (i.e., disease directed dosage). Fault tolerant and redundant computer systems will be used for data processing, to maintaining records relating to donor information and to ensure rapid and efficient retrieval stem cells from the storage repositories.

### 4. Stem Cell Banking

Collected and processed stem cells are “banked” for future use, at a stem cell bank or depository or storage facility, or any place where stem cells are kept for safekeeping. The storage facility may be designed in such a way that the stem cells are kept safe in the event of a catastrophic event such as a nuclear attack. In some embodiments, the storage facility might be underground, in caves or in silos. In other embodiments, it may be on the side of a mountain or in outer space. The storage facility may be encased in a shielding material such as lead.

a. I Unitized Storage

The physical steps of stem cell storage, including use of cryo-protectant (DMSO), controlled rate freezing and storage within a liquid nitrogen filled tank may comprise the prior art. The inventive aspect of the stem cell storage process is directed to the concept of unitized storage permits the storage of stem cells in multiple locations, either above or below ground. Such locations can be selected such that they are secure from physical events such as fires or earthquakes or other act of nature and from terrorist attack or acts of war. In addition, unitized storage facilitates the removal and use of only the necessary number of stem cell units for treatment, thus leaving other units for future use.

Specifically, unitized storage involves the banking of the harvested stem cells in separate storage bags of desired, defined units or dosages. At the time of use, only the required dosage is retrieved, by selecting the number of containers necessary to fulfill the desired dosage. Certain diseases may require stem cell therapy that includes a series of repeated treatments. By providing unitized storage of harvested stem cells, only the required dosage is retrieved for each treatment, to complete the entire therapy.

The number of units of stem cells for each storage container can be predetermined at random, in accordance with general prevailing stem cell therapy and treatment requirements, or in accordance with consideration of specific diseases anticipated to require stem cell therapy. For example, depending on the health condition, genetic history and/or profile of the donor, certain specific diseases may be targeted to potentially require or benefit from stem cell therapy in the future. Depending on the particular diseases targeted, the units required for each stem cell therapy treatment can be estimated before hand, so that each separate storage containers is filled with no more than the more likely amount to be used in the future. Each container does not necessarily contain the total amount expected to be used in a future treatment. The total amount of collected stem cells may be subdivided into defined fractional units in smaller containers, such that several containers of stem cells may be used to make up the total needed for a particular treatment.

Unitized storage for multiple dosage concept of the present invention is made possible only by the present invention, in that the inventive concept of elective collection and banking of autologous peripheral blood stem cells during pre-disease stage enables sufficient quantity and quality of harvested stem cells to be unitized into separate storage containers, each containing a prescribed number of units of stem cells. Generally, it may be desirable to bank at least 20 containers or units of stem cell for future stem cell therapy to treat certain diseases. Prior art allogeneic stem cell collection (e.g., from umbilical cords) simply does not result in sufficient quantity of stem cells, and certainly not in such quantity, and further not in a quality that would be effective.

Another inventive aspect of the invention is that each of the storage containers (e.g., bags or tubes) will be tagged with positive identification based on a distinctive property associated with the donor prior to storing in a stem cell bank. For example, DNA genetic fingerprint and HLA typing may be used with secured identification mechanism such as acceptable methods using microchips, magnetic strip, and/or bar code labels. This identification step 40 may be included in the process 30 in FIG. 3. Prior to use of the stem cells, a DNA sample is taken from the patient and compared to the DNA genetic fingerprint identification on the bags. This approach provides positive identification of the correct banked stem cells that originated from the particular patient.

### 5. Transplantation—Treatment Using Banked Stem Cells

Banked stem cells are applied to treatment of a patient who was the donor of the stem cells. Conventional standard transfusion methods (e.g. intravenous infusion) may be used for infusing the stem cells to a patient. Standard protocols for chemotherapy may be used followed by stem cells infusion for bone marrow reconstitution.

According to the present invention, the distribution of delivery of stem cells into a patient may be accomplished by any one of the conventional known infusion processes.

Normal conventional practice should be observed to monitor the progress of the patient undergoing transplantation. However, the applicants' method should reduce the amount of potential complications resulting from immune rejection, graft-versus-host-diseases, the duration of engraftment and infectious complications. The patient would benefit significantly because, if engraftment and reconstitution of the hematopoietic system does not occur after transplantation, the physician can rapidly detect this rejection and proceed with a second transplant.

### 6. An Embodiment of the Invention

Thus, in one embodiment of the invention: the stem cells of a non-neonate child or an adult (“person”), while the non-neonate child or adult is in a pre-disease state, are harvested and then preserved (such as cryopreservation). The harvesting (collection) process can be achieved using apheresis. There may be a need to infuse cell growth factors such as Granulocyte Colony Stimulating Factor, 1-6 days prior to the collection. To preserve the stem cells collected for future used, cryopreservation technique and reagent can be used.

Later (and this may be years later), should the same person develops cancer, an immunodisease, infectious disease, heart disease, brain disease, spiral cord disease, pancreatic disease, hepatic disease or bone marrow disease or undergoes therapy or is exposed to conditions which causes immunosuppression or infection or depletion of his immune cells, then the preserved stem cells or bone marrow are infused into the person to combat the disease. This can be achieved by intravenous infusion, intra arterial, intra-organ injection, intra bone marrow injection, intra-fat injection, intra-muscle injection of the stem cell products, or by intramedulary infusion (bone marrow), selective arterial infusion, pericardial infusion, epidural and subdural infusions. Similarly, the treatment protocol, and the criteria for determining the progress of the person and for adjusting the amount/dosage of cells to be infused can be achieved using standard transplantation practice.

Of course, the amount of stem cells collected should be sufficient for a major transplantation. If necessary, multiple collections should be done at an appropriate interval between collections (may be one week or more apart). However, as medicine advances, the preserved cells can be ex-vivo expanded and made to multiply or differentiate into the desired cell types before infusion into the person. If the person is deficient in certain subpopulation of cells, the subpopulation of cells from the preserved or expanded cells may be selected for in the future, and infused into the person. Furthermore, the harvested or expanded cells may be programmed by growing them in vitro with the person's diseased cells or tissues, or under stimulation by desired chemicals or cytokines before selecting for the desirable programmed cell and infusing them into the person.

In this embodiment, stem cells and bone marrow cells are chosen because they are versatile and because of their known use in cancer and immunodisease treatments and known methods for harvesting, processing, preserving, expanding them. Their use in such treatments may be employed in this invention. The following describes this embodiment in further details.

### 7. Example

By way of example and not limitation, an application of banked stem cells is described in reference to cancer treatment.

a. Development of Stem Cell Treatment of Cancer

Mechanisms that cause normal tissues to become malignant involve an “enormously complex process”*¹. Indeed, complexity in carcinogenesis occurs at each of many hierarchical levels. Even at the genetic level, tumor cells accumulate mutations in multiple genes during formation of most cancer types. Cancer is also the outcome of altered mechanisms occurring at other levels involving RNA, proteins, intracellular pathways, intercellular interactions, tissues, organs, etc. Since events occurring at one hierarchical level feed into and modify mechanisms at other levels, cancer development is a dynamic process that is more complicated than a simple summation of the parts. An important consideration is that some cancers may originate from or associated with stem cells*².

Thus, for cancer patients who face immunosuppressive therapy who have no readily matched donor, doctors have used “autologous” transplants: the cancer patient's bone marrow is removed, frozen, and stored prior to chemotherapy and/or radiation. Then the cells are thawed and reinfused into the patient after chemotherapy and/or radiation.

The collection of stem cell products (SC products), a term which includes both true stem cells and committed progenitor cells (i.e., CD 34+ cells are included), whether from bone marrow, cord blood or peripheral blood from third party donors, can be stored for future use, one of the most significant uses of stem cells is transplantation to enhance hematological recovery following an immunosuppressive procedure such as chemotherapy.

In the prior art, there is one significant drawback to the use of this very beneficial reinfusion procedure for treating a cancer patient. When SC products are obtained from the cancer patient, a significant number of tumor cells may also be collected, thereby contaminating the SC product.

Subsequently, when the SC product is reinfused into the cancer patient, the tumor cells are also reintroduced, increasing or re-introducing tumor cells into the patient's blood stream. While circulating tumor cells have not been directly linked to the relapse of a particular cancer, in the case of lymphoma, for example, reinfused cells have been traced to sites of disease relapse. In cases involving adenocarcinoma, it has been estimated that for a 50 kilogram adult, approximately 150,000 tumor cells can be reinfused during a single stem cell transplantation. Moreover, it has been shown that the tumor cells present in the SC product are viable and capable of in vitro clonogenic growth, thus suggesting that they could indeed contribute to post-reinfusion relapse. Ovarian cancer cells, testicular cancer cells, breast cancer cells, multiple myeloma cells, non-Hodgkin's lymphoma cells, chronic myelogenous leukemia cells, chronic lymphocytic leukemia cells, acute myeloid leukemia cells, and acute lymphocytic leukemia cells are known to be transplantable.

The extent of tumor cell contamination of SC products appears to vary greatly from patient to patient, and values within the range of 11 to 78 percent have been recorded. Therefore, as the reinfusion of circulating tumor cells may well circumvent the benefits provided by aggressive chemotherapy followed by stem cell transplantation*³.

Methods currently used to separate the valuable stem cells from the undesired tumor cell-contaminated product rely on positive or negative selection techniques. Positive selection assays identify stem cells and progenitor cells that express markers for the CD34 antigen and remove them from the blood or bone marrow product contaminated with tumor cells. These methods are very labor intensive, reduce the number of useable stem cells and require the use of specialized equipment, thus greatly increasing the cost of patient care and severely limiting the use of SC products in transplantation procedures. An alternative to positive selection for removal of tumor cells from blood was provided by Gudemann et al.*⁴, who described filtration with special leukocyte depletion membrane filters (which work by adsorbing charged particles) to remove urologic tumor cells from autologous blood during an intraoperative mechanical autotransfusion (IAT) procedure*⁴. A disadvantage of the membrane filters used by Gudemann et al is that they do not selectively retain tumor cells. White blood cells, including stem cells, are also retained. Thus, tumor cells are not removed from stem cells. The work of Miller et al also teaches that standard blood transfusion filters are ineffective at removing tumor cells from autologous blood.

On another front, in an attempt to improve the efficacy of stem cell therapy, scientists have exposed the cancer patients' extracted stem cells to modification in culture medium in the hope of “programming” them, such as to enhance their cancer fighting capability, before transfusing them into the patient. However, such studies have been unsuccessful in demonstrating a superiority of programmed stem cells versus native stem cells clinically.

b. Development of a Solution in Accordance with the Invention

Applicants see a need to improve the benefits of stem cell transfusion, which would ultimately result in increased survival rates, while at the same time providing a low-cost, clinically effective method for treating cancer patients with stem cell products. Prompted by such, applicants created the inventive methods disclosed herein based on certain initial hypotheses, which hypotheses may or may not be relevant to various embodiments of the inventive methods ultimately developed. Without wishing to be bound by the hypotheses postulated in this application, applicants made the following hypotheses. Each hypothesis may or may not relate to the other hypotheses. The efficacy of the invention in practice is obviously not bound by the correctness of the hypotheses.

As a first hypothesis, applicants believe that many cancers are systemic in nature at the time of diagnosis and widely distributed throughout the body. That is, applicants believe that by the time a patient has been diagnosed with many types of cancer, e.g., breast cancer, there can already exist cancer cells in other parts of the patient besides the perceived affected area. In fact, the cancer may have already spread or migrated throughout the patient's body, for example, as the cancer cells are being carried by the patient's circulating blood or lymphoid system. This hypothesis accounts for contamination of stem cells, by cancer cells, collected from the patient, which may cause relapse after transplantation.

As a second hypothesis, applicants believe that in some instances, malignant or pre-malignant cells are routinely generated by the human body. However, the human does not develop cancer because his normal cells “self-regulate” the body by monitoring and eliminating the malignant or pre-malignant cells before they proliferate uncontrollably and give rise to cancer. This is termed “immune surveillance”. Applicants further postulate that in some cancer patients, their previously healthy cells become diseased because their diseased cells have partially or completely lost the ability to “self-regulate” due to old age, and/or environmental assaults (exposure to radiation, carcinogens, or stress, etc.); and/or other factors as yet unknown. Thus, transplantation of such already diseased (defective) cells harvested from these patients may not be helpful.

As a third hypothesis, applicants believe that certain diseases arise due to the loss of one or more functions of a healthy cells, due to old age, and/or environmental assaults (exposure to radiation, carcinogens, or stress, etc.); and/or other factors as yet unknown. Such loss may result in the failure to self-regulate, or to generally sustain normal functioning of the body. For example, the cell loses the ability to produce an enzyme or chemical necessary for the body's proper functioning. Thus, such a loss may result in Alzheimer disease, Parkinson disease, etc.

In summary, based on the above hypotheses, even though the present application uses cancer as an example of a disease for treatment under the invention, it is understood that other diseases (which result from the partial or complete loss of one or more abilities of a cell over time; or a systemic disease; or immunodiseases, such as cancer) will similarly benefit from the present invention. Cancer is used herein merely for the convenience of illustration and discussion. The methods of the present invention can also be used to supplant immune cells to patients undergoing immunosuppressive treatments, such as chemotherapy, radiation therapy, or those who have been exposed to factors, which deplete their bodies of immune cells.

As a fourth hypothesis, the applicants believe that the programmed stem cells collected from cancer patients have not shown any observable advantage over unprogrammed stem cells from the same patient, because both the programmed and unprogrammed cells are already diseased and thus damaged. That is, both the programmed and unprogrammed cells have lost their cancer fighting ability (or their optimal cancer fighting ability as compared to healthy cells), and the “programming” cannot restore the normal function to the already diseased cells (which have irretrievably lost their function) necessary to fight cancer. Again, this hypothesis can be generalized to any disease, besides cancer, wherein a healthy cell will be more readily programmed that a diseased cell (which may be partially or completely unresponsive to programming).

c. Cancer Treatment

For ease of discussion, the following use breast cancer as a non-limiting example of cancer. The incident of breast cancer is the second highest, after lung cancer, in Caucasian women. Breast cancer is a difficult disease to treat. Patients undergoing chemotherapy, radiotherapy, or immunosuppressive therapy, generally lose immune cells. In the present invention, the patient's immune cells are replenished by his previously harvested pre-disease SC. Further, chemotherapy and radiotherapy destroy rapidly dividing cells which include cells found in bone marrow, the gastrointestinal tract (GI), and hair follicles. Thus, there is a threshold to the amount of chemical or radiation administered to the patient. Thus, with the stem cells replacement of this invention, a higher and more effective (aggressive) dose of chemotherapy or radiation may be administered to the patient to more aggressively eliminate the cancer cells.

### (i) Methods For SC Collection, Processing, Preservation and Infusion

Conventional methods for collecting, processing, cryopreserving, storing thawing, screening for and quantifying stem cells, and selecting for subpopulations of the stem cells, may be used.

Apheresis collection process of peripheral blood stem cells is a common method for collecting stem cells today. Hematopoietic cells can be isolated from human tissues including, for example, peripheral blood, bone marrow, fat tissue. Mononuclear cells, for example peripheral blood mononuclear cells (PBMCs) may be further isolated by methods such as density-gradient centrifugation. Sufficient quantity should be collected. If necessary, multiple collections should be considered to ensure enough dose for most demanding transplantation, typically about one (1) billion cells. The stem cells may be preserved by cryopreservation and later thawed for use, using standard transfusion procedures.

In an embodiment of the invention, all the stem cells collected can be cryogenically preserved, and used for hematopoietic reconstitution after thawing, in order to avoid cell losses associated with cell separation procedures*^{6?}. However, it is envisioned that cell separation procedures can be used if desired.

In one embodiment of the present invention for the primitive cell population to be further subdivided into isolated subpopulations of cells that are characterized by specific cell surface markers. The methods of the present invention may further include the separation of cell subpopulations by methods such as high-speed cell sorting, typically coupled with flow cytometry*⁷.

### (ii) Infusion and Transplantation

Conventional standard transfusion methods (e.g. intravenous infusion) may be used for infusing the stem cells. Standard protocols for chemotherapy may be used followed by stem cells infusion for bone marrow reconstitution.

### (iii) Confirmation that the Transplant is Working

Normal conventional practice should be observed to monitor the progress of the patient undergoing transplantation. However, the applicants' method should reduce the amount of potential complications resulting from immune rejection, graft-versus-host-diseases, the duration of engraftment and infectious complications. The patient would benefit significantly because, if engraftment and reconstitution of the hematopoietic system does not occur after transplantation, the physician can rapidly detect this rejection and proceed with a second transplant

Although the foregoing invention has been described in some detail by way of illustration and example for purposes of clarity and understanding, the above are by way of example, and are not meant to be limiting. It will be obvious that various modifications and changes that are within the skill of those skilled in the art are considered to fall within the scope of the appended claims.

Future technological advancements that allow for obvious changes in the basic invention herein are also within the claims.

All publications, figures, patents and patent applications cited herein are hereby expressly and fully incorporated by reference herein for all purposes to the same extent as if each had been fully set forth herein.

References

*¹ C. Sonnenschein, A. M. Soto, The Society of Cells-Cancer And Control of Cell Proliferation, BIOS Scientific Publishers Ltd and Springer-Verlag, New York, 1999

*² G. B. Pierce et al. Cancer—A Problem In Developmental Biology. Prentice Hall, New York, 1974, pp. 79-84; G. B. Pierce, in The Biological Basis of Cancer, R. G. McKinnel, R. E. Parchment, A. O. Perantoni, G. B. Pierce, Eds., Cambridge Univ. Press, Cambridge UK, 1998, pp. 39-47

*³ United States Patent Application, Publication No. 2001 0000204 A1, of Castino et al., published Apr. 12, 2001. Hereinafter referred to as “Castino et al.

*⁴ Gudemann, C., Wiesel, M. And Staehler, G., Intraoperative Autotransfusion In Urologic Cancer Surgery By Using Membrane Filters, XXIII.sup.rd Congress of the ISBT, abstracts in Vox Sang., 67 (S2), 22.)

*⁵ Miller, G. V., Ramsden, C. W. and Primrose, J. N., Autologous transfusion: an alternative to transfusion with banked blood during surgery for cancer, B. J. Surg. 1991, Vol. 78, June, 713-715

*⁶ Douay et al., 1986, Recovery of CFU-GM from cryopreserved marrow and in vivo evaluation after autologous bone marrow transplantation are predictive of engraftment. Exp Hematol. 14(5):358-365; Knight, 1980, Preservation of Leukocytes in Low Temperature Preservation in Medicine and Biology, Ch. 6, Ashwood-Smith and Farrant (University Park Press, Baltimore) pp. 121-137.

*⁷ United States Patent Application Publication No. 20020146680 A1, of Rich, Ivan N., published Oct. 10, 2002, entitled “High-throughput stem cell assay of hematopoietic stem and progenitor cell proliferation.

What is claimed is:

1. A process of making stem cells available to a person, comprising the steps of:
the person proactively electing to have his stem cells collected with no immediate perceived health condition requiring treatment using his own collected stem cells;
collecting stem cells from the person;
at the time of collection, earmarking the collected stem cells for use by the person;
preserving the collected stem cells in storage; and
retrieving the stored stem cells if and when needed by the person.

2. The process of claim 1, wherein the electing step includes the step of considering a targeted disease or a class of diseases to which the collected stem cells are intended to be applied for treatment.

3. The process of claim 2, wherein the collecting step is undertaken at a time when the person is in a pre-disease stage, including at least the stage in which the person has not been diagnosed of the targeted disease or diseases to which the collected stem cells are intended to be applied for treatment.

4. The process of claim 3, wherein the collecting step collects stem cells in sufficient quantity that is anticipated to be needed for such treatment.

5. The process of claim 1, wherein the collecting step is conducted when the person is an adult or a non-neonate child.

6. The process of claim 1, wherein the collecting step is conducted when the person met at least one of a prescribed weight and age.

7. The process of claim 6, wherein the collecting step is conducted when the person is met at least one of the following conditions: between 10-200 Kg. weight and between 2 to 80 years old.

8. The process of claim 1, wherein the collecting step is undertaken over multiple sessions, at at least one of different ages and weights of the person.

9. The process of claim 8, wherein the collecting step includes the step of collecting at least on the order of 10⁶ total nucleated cells per Kg. weight of the person in a single collection session.

10. The process of claim 1, wherein the preserving step comprises storing the collected stem cells in a stem cell bank.

11. The process of claim 10, wherein the preserving step comprises the step of processing the stem cells, including unitizing the collected stem cells into multiple separate units of containers.

12. The process of claim 11, wherein the processing step comprises the step of optimizing at least one of the number of units and the number of stem cells in each unit prior to storing in the stem cell bank, with consideration of intended disease or diseases to which the stem cells will be applied.

13. The process of claim 12, wherein each unit comprises less than on the order of 10⁶ total nucleated cells per Kg. weight of the person.

14. The process of claim 13, wherein each unit comprises a dosage of a fraction of the total nucleated cells required to be applied to the intended disease.

15. The process of claim 14, wherein the units contain equal or different dosages.

16. The process of claim 10, wherein the preserving step is independent of tissue or HLA typing of the collected stem cells prior to storing in the stem cell bank.

17. The process of claim 11, wherein the preserving step comprises the step of determining from the collected stem cells at least a distinctive property associated with the person prior to storing in a the stem cell bank, so as to provide a means of secured identification to match the collected stem cells with the person at the time of use.

18. The process of claim 17, wherein the typing step includes providing an indicia with each unit representing information of said at least one distinctive properties.

19. The process of claim 18, wherein the indicia is embodied in at least one of a label, bar code, magnetic strip, and microchip.

20. The process of claim 3, wherein the stem cells are collected during a pre-disease stage in which the person may be diagnosed with a health condition that is not similar to the disease to which the collected stem cells are intended to be applied for treatment.

21. A process for treatment of a person, comprising the steps of
the person proactively electing to have his stem cells collected with no immediate perceived health condition requiring treatment using his own stem cell;
collecting stem cells from the person;
at the time of collection, earmarking the collected stem cells for use by the person; preserving the collected stem cells in storage;
retrieving the stored stem cells if and when the person is diagnosed with a disease requiring stem cell treatment; and
treating the person using his own stem cells retrieved from storage.

22. The process as in claim 21, wherein the electing step includes considering a targeted disease or a class of diseases to which the collected stem cells are intended to be applied for treatment, and wherein at the time of collection, the person is not diagnosed with such targeted disease or diseases.

23. The process as in claim 22, wherein the collecting step includes collects stem cells in such quantity as to be sufficient in anticipation of the treatment.

24. The process as in claim 21, further comprising the steps of:
determining from the collected stem cells at least a distinctive property associated with the person prior to storing in a the stem cell bank, so as to provide a means of secured identification to match the collected stem cells with the person at the time of use; and
at the time of use of the stored stem cells, matching the distinctive property with a sample from the person to positively identify the stored stem cells as being collected from the person.

25. A process for treatment of a person, comprising the steps of:
the person proactively electing to have his stem cells collected with no immediate perceived health condition requiring treatment using his own stem cell;
collecting stem cells from the person;
applying the collected stem cells after the person has been diagnosed with a disease requiring stem cell treatment; and
treating the person using his own collected stem cells.
